(19) United States
(12) Patent Application Publication
(10) Pub. No.: US 2004/0258753 A1
(43) Pub. Date: Dec. 23, 2004

(54) Pulsed bio-agent delivery systems based on degradable polymer solutions or hydrogels

(76) Inventors: Jo Demeester, Sint-Amandsberg (BE); Stefaan De Smedt, Mariakerke (BE); Barbara Stubbe, Balegem-Oosterzele (BE); Bruno De Geest, Sint-Denijs-Westrem (BE)

Correspondence Address: CLARK & ELBING LLP, 101 FEDERAL STREET, BOSTON, MA, 02110, US

(21) Appl. No.: 10/871,959

(22) Filed: Jun. 18, 2004

Related U.S. Application Data
(63) Continuation-in-part of application No. PCT/BE02/00195, filed on Dec. 20, 2002.

(30) Foreign Application Priority Data
Dec. 21, 2001 (GB) 0130518.4

(51) Int. Cl. A61K 9/24

(52) U.S. Cl. 424/473

(57) ABSTRACT

A degradable oligomer or polymer aqueous solution or a degradable polymer hydrogel, wherein degradation occurs by cleavage of the oligomer or polymer backbone and/or, in the case of a hydrogel, by cleavage of cross-linking bonds within the hydrogel, is useful as a component of a time-controlled explosion bio-agent release system or a pulsed bio-agent delivery system comprising at least one biologically active agent and an outer semi-permeable lipid or polymer membrane, wherein bio-agent release or delivery begins after a lag time.

### CROSS REFERENCE TO RELATED APPLICATIONS

This application is a continuation-in-part of International Application No. PCT/BE2002/00195, filed on Dec. 20, 2002, which was published in English under PCT Article 21(2), and which claims the benefit of British patent application No. 0130518.4 filed on Dec. 21, 2001, the disclosures of which are incorporated by reference in their entirety.

The present invention relates to the time-controlled delivery of bio-agents such as therapeutic drugs, proteins, vitamins, hormones, biocides, pesticides and the like. More precisely, the invention relates to the use of degradable polymer solutions or hydrogels for time-controlled or pulsed bio-agent release or delivery systems. In particular, the invention relates to such systems comprising a semi-permeable membrane and a bio-agent containing core, the composition and structure of which allows for single or multiple pulse delivery of the bio-agent.

### BACKGROUND OF THE INVENTION

Currently, there is a major interest in pulsed drug delivery in which the pharmaceutical device releases the drug at a pre-programmed time. Pulsed drug release can be achieved in different ways, namely by creating a rigid, semi-permeable membrane around a core comprising the drug and a swellable component. The role of such a membrane is (i) to allow for the transport of small molecules (e.g. water molecules, ions) between the swellable component and the surrounding solution, and (ii) to prevent larger molecules (e.g. proteins, polymeric degradation products) to leave the device. During time, the swelling pressure nsw of the core gradually increases. When π_{sw} exceeds the tensile strength of the membrane, the core ruptures, followed by a sudden release of the drug. This is illustrated for instance in U.S. Pat. No. 4,871,549 disclosing a so-called time-controlled explosion system in which drug release is caused by explosion of an outer membrane after a definite time period (defined as a “lag time ”) which can be controlled by the sort or amount of swelling agent and membrane. The drug delivery system of U.S. Pat. No. 4,871,549 may be in the form of beads or granules, wherein for instance sucrose granules are coated with an acidic or basic drug (e.g. with a spraying binder dissolved in a suitable solvent), then a swelling agent (which may be a disintegrating agent, a synthetic polymer, an inorganic or organic salt) or effervescent agent is coated on the drug-coated granules, and finally the swelling agent-coated granules are coated with a water-insoluble coating material such as ethyl cellulose to form the outer membrane. The proportions of drug and swelling agent in the beads or granules are preferably 0.1 to 50 and 30 to 80 weight percent respectively. The drug delivery system of U.S. Pat. No. 4,871,549 may also be in the form of a tablet prepared by compressing a mixture of drug, swelling agent, diluent and lubricant, the proportions of drug and swelling agent in the tablet preferably being 0.1 to 30 and 10 to 60 weight percent respectively, and finally coating the tablet with a water-insoluble coating material such as ethyl cellulose to form the outer membrane. By mixing such systems having different lag times, various release patterns may be achieved such as repeat pattern, zero-order pattern (i.e. uniform rate release), reverse first-order pattern (i.e. release rate increases with time) or a sigmoid pattern. However U.S. Pat. No. 4,871,549 makes no suggestion of using a degradable hydrogel for making a time-controlled explosion system.

Other systems are also known in the art. For instance, U.S. Pat. No. 3,247,066 discloses a core comprising a mixture of drug and a water-swellable colloid coated with a water-permeable polymer. When used for oral administration, body fluid water permeates the coating, causing the colloid to hydrate and swell and break the outer coating thus releasing the drug. This device however suffers from the inherent defect that the swelling of colloids is greatly influenced by pH. U.S. Pat. No. 3,952,741 also discloses an osmotic dispenser wherein a water-permeable membrane surrounds an active agent optionally mixed with an osmotic attractant. U.S. Pat. No. 4,933,185 discloses a controlled release system comprising microcapsules having an inner polysaccharide core and an outer ionically interacting skin, a biologically active substance, and an enzyme specifically degrading said core polyssacharide, not the ionically interacting skin, until the outer skin loses its integrity and the microcapsules completely break down. U.S. Pat. No. 5,593,697 discloses an implant for parenteral administration comprising (i) a drug preferably contained in a core, (ii) an excipient system comprising one water-soluble, preferably biodegradable, material (e.g. lactose) and one water-insoluble, preferably swellable or disintegrating, material (e.g. sodium starch glycolate or stearic or palmitic acid), and (iii) a polymer film coating adapted (e.g. by the incorporation of a permeability modifying agent such as hydroxypropylmethyl cellulose) to rupture after a lag time, the said outer film being impermeable to peptides, proteins, antigens and the like. In the latter embodiment, the lag time is controlled by varying the thickness of the outer film or by the amount of hydroxypropylmethyl cellulose in the coating film.

In the various drug delivery systems described above, the swelling agents used are non-degradable. There is a need in the art for drug delivery devices with more predictable release profiles. There is also a need in the art for drug delivery devices taking advantage of the biodegradability, hence bio-compatibility, of some of their components. The purpose of the present invention is to address these problems.

Hydrogels are well suited for biomedical applications because of their bio-compatibility, however degradable hydrogels have not yet been proposed as swelling controlled drug delivery components.

Furthermore, U.S. Pat. No. 5,654,006 discloses a composition for parenteral administration, including encapsulated microparticles having an average size between 0.05 and 5 μm, for rapid release of a therapeutic compound when the composition is exposed to a selected target condition related to pH, temperature or the presence of a selected ligand. The microparticle and entrapped drug are encapsulated within a lipid bilayer membrane. Localized disruption of the lipid membrane, and influx of monovalent ions into the polymer matrix, in response to the selected target conditions, causes a cascade effect involving matrix swelling and further membrane disruption. This composition however suffers from the limitation that a change in ionic environment is required for membrane disruption. U.S. Pat. No. 6,537,584 discloses blends of chitosan (a cationic polymer) and a second polymer that, once hydrated, are substantially insoluble in acid or, if soluble, remain rigid in acidic conditions for a sufficient period of time to modulate drug delivery. However, U.S. Pat. No. 6,537,584 makes no suggestion of a degradable hydrogel or an outer membrane or a time-controlled delivery.

International patent application published as WO 97/04747 discloses pharmaceutical formulations made by entrapping a drug in either an organic or water phase biodegradable hydrogel polymer system to produce nanoparticles with a sphere size typically in a range of 500 to 1,500 nm which are then coated or combined with a bioadhesive adjuvant to promote adherence to the intestinal wall. These nanoparticles can be absorbed by the body by the lymphatic or lacteal system and, because of the resistant hydrogel coating, are not affected by enzymes present in the gastrointestinal tract. Suitable bioadhesive adjuvants disclosed in WO 97/04747 are hydroxypropyl methyl cellulose, methyl cellulose, pectin, guar gum, xantham gums, gum acacia, gum dragon, hydroxypropyl alginate, sodium carboxymethyl cellulose, carbomer 934-P and acrylic acid derivatives. WO 97/04747 makes no suggestion of a semi-permeable membrane for pulsed drug delivery.

Fransen et al. in Journal of Controlled Release (1999) 60:211-221 describe the controlled release of a protein from a protein-loaded hydroxyethyl methacrylated dextran hydrogel, wherein hydrogel degrada-bility under physiological conditions was due to the presence of hydrolytically sensitive carbonate esters in the crosslinks of the gels. This reference however makes no suggestion of using a semi-permeable membrane for pulsed drug delivery. Gennaro in The Science and Practice of Pharmacy (2000), chapter 47, pages 912-913, discloses a tablet consisting of a core of an osmotically active drug, or an osmotically inactive drug in combination with an osmotically active salt surrounded by a semi-permeable membrane. The advantage of this osmotic system is that it requires only osmotic pressure to be effective and is essentially independent of the environment. This reference however makes no suggestion of using a degradable polymer hydrogel for pulsed drug delivery.

One problem addressed by the present invention is to provide a bio-agent, e.g. a drug, delivery system based on membrane disruption wherein the latter occurs independently from any change in the biological environment.

### SUMMARY OF THE INVENTION

The present invention is based on the principle that in a bio-agent delivery or release system comprising a semi-permeable membrane surrounding a core comprising said bio-agent (e.g. drug) and a swellable component, the lag time may be suitably controlled by the design and proper selection of an in situ, e.g. in vivo, degradable swelling agent rather than by the complicated procedures of tailoring some features, such as composition and thickness, of the membrane, or by only tailoring such features. For the proper development of such delivery devices with predictable release profiles a detailed understanding of the thermodynamic and kinetic properties of degrading hydrogel systems is required.

Based on this underlying principle, the present invention firstly provides the use of a degradable oligomer or polymer in the form of an aqueous solution or a hydrogel, wherein degradation occurs by cleavage (i.e. usually hydrolysis) of the oligomer or polymer backbone and/or, in the case of a hydrogel, by cleavage (i.e. usually hydrolysis) of cross-linking bonds within the said hydrogel, as a component of a time-controlled explosion bio-agent release system or a pulsed bio-agent (biologically active agent) delivery system comprising at least one bio-agent and an outer semi-permeable lipid or polymer membrane, wherein the bio-agent release or delivery begins after a lag time.

Secondly, the present invention provides a time-controlled explosion bio-agent release system or pulsed bio-agent delivery system comprising at least (i) an outer semi-permeable membrane wherein the bio-agent release or delivery is caused by disruption or explosion of the said membrane and begins after a lag time and at least (ii) a core comprising a bio-agent and a swelling agent responsible for the disruption or explosion of said semi-permeable membrane, said release or delivery system being characterised by the fact that the said swelling agent is a degradable oligomer or polymer in the form of an aqueous solution or a hydrogel, wherein degradation occurs by cleavage of the polymer backbone and/or, in the case of a hydrogel, by cleavage of cross-linking bonds within the said hydrogel.

### BRIEF DESCRIPTION OF THE DRAWINGS

FIG. 1 shows the variation of the amount of free dextran as a function of time in degrading hydrogels of dextran modified by means of hydroxyethyl methacrylate (hereinafter referred to as dex-HE A) with various degrees of substitution (i.e. number of HEMA groups per 100 glucopyranose residues of dextran, hereinafter referred to as DS) and various concentrations.

FIG. 2 shows the variation of the elastic modulus G′ as a function of time in degrading dex-HEMA hydrogels with various DS and concentrations.

FIG. 3 shows the swelling pressure π_{sw} as a function of the polymer volume fraction φ of dex-HEMA hydrogels with various DS and concentrations, before degradation.

FIG. 4 shows the swelling pressure π_{sw} of a dex-HEMA hydrogel de-swollen in a polyethylene glycol solution after certain periods of time as a function of the polymer volume fraction φ.

FIG. 5 shows the variation, as a function of degradation time t, of the constant A in the equation of Horkay et al. (see hereunder) linking the swelling pressure π_{sw} to the polymer volume fraction (p in a dex-HEMA hydrogel.

FIG. 6 shows the variation of the swelling pressure π_{sw}, as a function of degradation time t, for two types of dex-HEMA hydrogels.

FIG. 7 shows the variation of the swelling pressure π_{sw}, as a function of degradation time t, of a methacrylated dextran (hereinafter referred to as dex-MA) hydrogel during degradation by dextranase.

FIG. 8 shows the variation of the osmotic pressure, as a function of time, of buffer diluted solutions of degrading diblock and triblock copolymers of lactic acid and polyethyleneglycol.

FIG. 9 shows the variation of the osmotic pressure, as a function of time, of a buffer diluted solution of degrading γ-cyclodextrin.

FIG. 10 shows the size distribution of dex-HEMA microgels used in an embodiment of this invention.

FIG. 11 shows the zeta-potential of negatively and positively charged dex-HEMA microgels used in an embodiment of this invention.

FIG. 12 shows the zeta-potential of uncoated dex-HEMA microgels.

FIG. 13 shows the zeta-potential of layer-by-layer coated dex-HEMA microgels used in an embodiment of this invention.

FIG. 14 shows scanning electron microscopy images of both uncoated microgels (upper part) and layer-by-layer coated microgels (lower part).

### DETAILED DESCRIPTION OF THE INVENTION

The present invention provides the use of a degradable oligomer or a degradable polymer aqueous solution or a degradable oligomer or polymer hydrogel as a component of a time-controlled explosion bio-agent release system or a pulsed bio-agent delivery system comprising at least one biologically active agent and an outer semi-permeable lipid or polymer membrane, wherein bio-agent release or delivery begins after a predetermined time (so-called “lag time”). Depending upon the construction of the release or delivery system, i.e. in particular depending upon the selection of the particular in situ, e.g. in vivo, degradable oligomer or polymer, the selection of the particular bio-agent, the bio-agent (e.g. drug) loading and the intended route of administration, the lag time may vary within extremely broad ranges from about one hour to two weeks, preferably from about 1 to 24 hours.

Basically in situ, e.g. in vivo, degradation of the degradable oligomer or polymer aqueous solution or hydrogel occurs by cleavage (i.e. usually hydrolysis) of the polymer backbone or by cleavage (i.e. usually hydrolysis) of cross-linking bonds (usually covalent bonds) within the hydrogel, depending on the chemical type of the said oligomer or polymer. Whatever the degradation mechanism may be, the lag time, i.e. the time period after which the membrane ruptures, is at least partially, preferably mainly, and more preferably substantially completely controlled by the degradation rate of the said degradable oligomer or polymer aqueous solution or hydrogel. In other words, other parameters of the delivery system such as the composition and thickness of the membrane, although they may be modified in order to improve the system efficiency while using general knowledge of those skilled in the art, are not critical to the present invention and therefore will not be discussed in detail herein. Although this parameter is not a requirement of the invention, the concentration of the degradable oligomer or polymer in the aqueous solution may be, depending on the specific nature of said oligomer or polymer and on the specific nature of the semi-permeable membrane, within a range from about 1% to about 40% by weight, preferably from about 5% to about 30% by weight.

The in situ, e.g. in vivo, degradable oligomer or polymer for use in this invention may be any linear or branched water-soluble polymer backbone having at least two termini, at least one of the said termini being optionally covalently bonded to a linker, wherein at least one of the polymer backbone and the linker comprise a hydrolytically or enzymatically degradable linkage. The term “linkage” or “linker” is used herein to refer to groups or bonds that normally are formed as the result of a chemical reaction and typically are covalent linkages. Hydrolytically degradable linkages means that the linkages are degradable in water or in aqueous solutions at useful pHs, e.g. under physiological conditions, including for example in blood. Enzymatically degradable linkages means that the linkage can be degraded by one or more enzymes. For instance, the in vivo degradable oligomer or polymer for use in this invention may be a polypeptide, provided that said polypeptide be in vivo degradable by an enzyme, such as a protease, which is present in the part of the body where membrane disruption is desired.

In a first specific embodiment of this invention, the degradable oligomer or polymer may be selected from the group consisting of disaccharides (such as for instance sucrose), oligosaccharides (such as for instance p-nitrophenyl-penta-N-acetyl-chitopentaoside) and polysaccharides, all of them being enzymatically cleavable. Such di-, oligo- and polysaccharides may have any molecular weight ranging from about 100 to about 1,000,000, preferably from about 200 to about 200,000, and more preferably from about 1,000 to 40,000. Although the invention is not limited to such a kind of in vivo degradable polymer hydrogels, the present invention will now be explained in more detail by reference to degradable modified dextran hydrogels which are enzymatically cleavable by dextranase. As is well known, dextran is a high molecular weight (about 15,000 to 150,000) polysaccharide containing α-glucopyranose units which may be produced from the action of Leuconostoc mesenteroides onto saccharose. Dextran may be chemically modified by reaction with functional α-β ethylenically unsaturated acid esters such as functional acrylates and methacrylates. For instance, methacrylated dextran (hereinafter referred to as dex-MA) may be obtained by coupling glycidyl methacrylate to dextran, as disclosed by Van Dijk et al. in Macromolecules (1995) 28:6317-6322. Dextran may also be modified by one or more (C₁₋₈ alkyl) acrylate or methacrylate by reacting dextran with an epoxy (meth)acrylate such as the superior homologues of glycidyl acrylate or methacrylate. Within the polymer aqueous solutions or hydrogels of such (meth)acrylated dextrans, degradation occurs by cleavage of the polymer backbone, more specifically by the enzymatic action of dextranase and/or by hydrolysis of the carbonate ester link formed between the methacrylate group and the dextran molecule.

Dextran may also be modified by means of at least one (hydroxy-C₁₋₈ alkyl) acrylate or methacrylate, such as for instance hydroxyethyl methacrylate, thus leading to a structure which may be represented by the following formula:

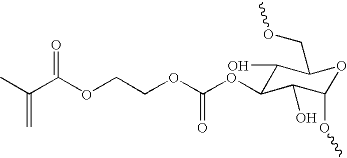

Dextran may also be modified by means of other α,β-ethylenically unsaturated entities, such as for instance acrylamides and methacrylamides, provided that that the bonds thus formed are degradable.

In polymer solutions and hydrogels from the latter modified dextran, the formation of covalent cross-linking bonds is a common feature and, hence, degradation occurs mainly by cleavage (hydrolysis) of the said cross-linking bonds within the hydrogel.

Preferably the degree of substitution (i.e. the number of chemically modifying groups, e.g. (meth)acrylic groups, per 100 glucopyranose residues of dextran) of the modified dextran used in this invention is between about 2 and 10.

After the chemical modification of dextran, the resulting modified dextran may be dissolved in a buffer at a suitable pH, e.g. usually a pH between about 6.5 and 8.5, and the resulting aqueous solution may then be radically poly-merized in the presence of a suitable soluble catalyst or catalytic system comprising, for example, N,N,N′,N′-tetramethylene-ethylenediamine (hereinafter TEMED) and potassium persulfate (hereinafter KPS) until a hydrogel is obtained. Gelation can also be obtained by photopolymerisation in the absence or in the presence of a photo-initiator. Catalysts and photo-initiators suitable for this purpose are well known in the art.

Other examples of degradable oligomers or polymers that are suitable for the present invention include cyclodextrins and modified cyclodextrins that are enzymatically cleavable by amylase. Cyclodextrins and modified cyclodextrins, in particular their pharmaceutical grades, are well known in the art and are available from a variety of commercial sources. They may be collectively referred as starch cyclic degradation products containing 6 to 8 glucose residues, or alternatively as cyclic oligosaccharides composed of L-glucose molecules linked by α or β osidic bonds having a toric form. A suitable representative embodiment of modified cyclodextrins consists of hydroxypropyl-β-cyclodextrin.

Other examples of degradable oligomer or polymer hydrogels that are hydrolylitically degradable and thus suitable for carrying out the present invention include, for instance, hydrogels based on synthetic polymer backbones from substantially non-immunogenic polymers, such as polyether polyols, including those with two or more hydroxyl groups derived from polyethylene glycol (PEG) or a copolymer of ethylene oxide and an alkylene oxide (e.g. propylene oxide) with a degree of polymerization up to about 500. For instance the sequence present in the said polyether polyol may be represented by the formula —O—R—O—, wherein R may be an alkylene group possibly substituted with one or more hydroxy groups or alternatively R may be

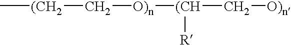

wherein R′ is alkyl group with up to 4 carbon atoms, preferably methyl, n is an integer up to about 200, and n′ is an integer up to about 100. However, it should be understood that other related polymers are also suitable for use in the practice of this invention. PEG is typically clear, colorless, odorless, soluble in water, stable to heat, inert to many chemical agents, does not hydrolyze or deteriorate and is generally non-toxic. Poly(ethylene glycol) is considered to be biocompatible, which is to say that PEG is capable of coexistence with living tissues or organisms without causing harm. More specifically, PEG is non-immunogenic, which is to say that PEG does not tend to produce an immune response in the body. When attached to a molecule having some desirable function in the body, such as a biologically active agent, the PEG tends to mask the agent and can reduce or eliminate any immune response so that an organism can tolerate the presence of the agent. PEG conjugates tend not to produce a substantial immune response or cause clotting or other undesirable effects. PEG preferably PEG having a molecular weight of from about 200 to about 100,000 may suitably be used as the polymer backbone and is therefore one useful polymer in the practice of the invention.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched polyethylene glycols can be represented in general form as R(--PEG--OH)ₘ in which R represents the core moiety, such as glycerol, pentaerythritol or sorbitol, and m is an integer which represents the number of branches. Other suitable embodiments include PEG coupled to polylactic acid or polyglycolic acid in the form of diblock copolymers or triblock copolymers.

Many other polymers are also suitable for the present invention. These polymers can be either in linear form or branched form, and include in their structure, but are not limited to, other poly(alkylene glycol), such as poly(propylene glycol), copolymers of ethylene glycol and propylene glycol and the like, poly(oxyethylated polyol), poly(olefinic alcohol), poly(vinylpyrrolidone), poly (hydroxypropylmethacrylamide), poly(α-hydroxy acid), poly(vinyl alcohol), poly-phosphazenes, polyoxazolines; polymers and copolymers (whether random, block, segmented or grafted) of lactones such as ε-caprolactone, glycolide, L-lactide, D-lactide, meso-lactide, 1,4-dioxan-2-one, trimethylene carbonate (1,3-dioxan-2-one), χ-butyrolactone, δ-valerolactone, 1,5-dioxepan-2-one, 1,4-dioxepan-2-one, 3-methyl-1,4-dioxan-2,5-dione, 3,3 diethyl-1,4-dioxan-2,5-one, ε-decalactone, pivalolactone and 4,4-dimethyl-1,3-dioxan-2-one and the like; Several embodiments of such copolymers have been described by, among others, U.S. Pat. No. 5,951,997, U.S. Pat. No. 5,854,383 and U.S. Pat. No. 5,703,200 and shall be considered as being within the scope of the present invention; hydroxy-terminated polyorthoesters obtainable for instance by the addition reaction of a diol (e.g. an alkylenediol such as ethylenediol, trimethyleneglycol, tetramethyleneglycol, pentamethyleneglycol, hexanediol-1,6 and the like, or a cycloalkyldiol such as 1,4-cyclohexanedimethanol or 1,4-cyclohexanediol) or polyethyleneglycol onto a diketene acetal; such a method for a hydroxy-terminated polyorthoester is well known in the art and is described, starting from 3,9-bis(ethylidene-2,4,8,10-tetraoxaspiro[5,5] undecane, by J. Heller et al. in Macromolecular Synthesis 11: 23-25; Hydroxy-terminated polyacetals obtainable for instance by the condensation reaction of at least a diol (such as hereinabove mentioned) and a divinylether as is well known in the art; for instance, U.S. Pat. No. 4,713,441 describes unsaturated, linear, water-soluble polyacetals having molecular weights from about 5,000 to about 30,000 which may be formed by condensing a divinylether, a water-soluble polyglycol and a diol having a (preferably pendant) unsaturation, which may be further converted to hydrogels, for instance by using a free-radical initiator in order to copolymerize the double bonds in the polyacetal with a monomeric compound having a reactive double bond. Another typical procedure for this kind of polyacetals may be found in Heller et al., Journal of Polym. Science, Polym. Letters Edition (1980) 18 :293-7, starting from 1,4-divinyloxybutane or diethyleneglycol divinylether. French patent No. 2,336,936 further refers to crosslinked polyacetals formed by condensing diols or polyols with 3,4-dihydro-2H-pyran-2-ylmethyl-3,4-dihydro-2H-pyran-2-ylcarboxylate.

Other examples of degradable oligomer or polymer hydrogels that are hydrolylitically degradable and thus suitable for carrying out the present invention also include macromers based on the above mentioned synthetic polymer backbones and further including one or more poly-merizable region(s) containing for instance polymerizable end groups such as ethylenic and/or acetylenic unsaturations. The choice of said polymerizable end groups will be dictated by the need for rapid polymerization and gelation. Therefore, namely because they can easily be polymerized while using various polymerization initiating systems, as is well known in the art, vinyl groups such as but not limited to acrylate, methacrylate, acrylamide and methacrylamide groups are preferred.

Although the molecular weight of each chain of the polymer backbone can vary, it is typically in the range of from about 100 to about 100,000, preferably from about 6,000 to about 80,000.

Those of ordinary skill in the art will recognise that the foregoing list for non-immunogenic polymer backbones is by no means exhaustive and is merely illustrative, and that all polymeric materials having the qualities described above are contemplated.

This polymer solution or hydrogel is then ready to be used as a component of a time-controlled explosion bio-agent release system or a pulsed bio-agent delivery system comprising at least one biologically active agent and an outer semi-permeable lipid or polymer membrane. The term “semi-permeable”, as used herein, means a membrane which is permeable to ions and water, but impermeable to the bio-agent and the degradation products. Such semi-permeable membranes are well known in the medical art, being useful namely for dialysis. They may be made from cellulose (either natural or regenerated) by dissolving it in special inorganic solvents (e.g. the so-called cupro-ammonium process) and reforming the polymer by removing the solvent to form flat sheet, tubular or hollow fibre membranes. Their molecular weight cut off may range from about 1,000 to 50,000, preferably from about 5,000 to 20,000. They are commercially available from a number of suppliers such as, but not limited to, Visking, Medicell and the like, including commercial grades such as Cuprophan®.

In another embodiment, the invention refers to the use of a hydrogel or microgel such as above described, the said hydrogel or microgel being coated with a lipid, for making a pulsed delivery system such as defined hereinbefore. This system is thus based on the degradation of a hydrogel core surrounded with a lipid coating layer. A suitable hydrogel used in the following illustration of this embodiment is a hydroxethyl methacrylated dextran with a M_{w} of 19,000 g/mole, but the invention is not limited thereto.

Lipid coatings, e.g. lipid bilayers, are ideally suited as a surrounding membrane for the degradable polymer hydrogels or microgels of this invention due to the restricted permeability of such coatings. They are permeable to water but impermeable to the degradation product of the hydrogel and the encapsulated drug. Any lipid or lipid mixture suitable for making liposomes may be conveniently used in this embodiment of the invention. Although lipid coating spherically shaped hydrogels has already been reported in literature, it is not a straightforward technique. Several attempts dealt with the problem of incomplete coating efficiency and unless a fatty acid layer is covalently attached to the gel surface, successful coating yielding 100% efficiency cannot be achieved.

This problem has now been solved by the present invention, wherein by using electrostatic interactions it becomes possible to achieve 100% coating efficiency. By incorporating suitable co-monomers such as, but not limited to, dimethylaminoethyl methacrylate (DMAEMA; e.g. one mole per mole HEMA), respectively methacrylic acid (MAA; e.g. one mole per mole HEMA) into the degradable polymer microgels of this invention, the latter become positively (dex-HEMA-DMAEMA), respectively negatively (dex-HEMA-MAA), charged. A 100% lipid-coating efficiency was then achieved by immersion of the thus modified microgels into a solution containing lipid vesicles (e.g. 1 mg/ml) oppositely charged to the microgels, e.g. using the well known Layer-by-Layer Electrostatic Self Assembly (LbL-ESA) procedure. The same successful result can be obtained with neutrally charged degradable polymer microgels, provided that a polyelectrolyte layer was put onto the microgels in order to create a charged template. When the lipids used are insufficiently soluble in water, they may be first dissolved (e.g. 2 mg/ml) in chloroform. Subsequently chloroform may be evaporated to yield a lipid film. In order to obtain suitable lipid vesicles, water may be added up to a final lipid concentration of e.g. 1 mg/ml. It is assumed that nanoscopic lipid vesicles (i.e. liposomes) adsorb onto the degradable polymer microgel by electrostatic interaction. Once the surface of said microgel is covered, the lipid vesicles spread and form a lipid layer. The success of this procedure was proven using confocal laser scanning microscopy (CSLM) and measuring the electrophoretic mobility of the particles before and after coating. It was also determined whether the swelling pressure of such dex-HEMA gels is sufficient to rupture such a lipid coating by measuring the tensile strength of the lipid membranes using a carboxy fluoresceine release method on 100 nm sized lipid vesicles. Table 1 gives an overview of the charge, tensile strength and the critical swelling pressure for different lipid compositions. This table indicates that dex-HEMA gels are indeed able to rupture a lipid membrane upon degradation of dex-HEMA microgels with a mean diameter of about 3 μm.

TABLE 1

| | Molare | | T_{crit} | [symbol] _{swell,crit} |
|---|---|---|---|---|
| Lipid components | ratio | charge | [N/m] | [kPa] |
| SOPC:DOTAP | 9:1 | + | 0.066 | 88 |
| SOPC:DOTAP | 7:3 | + | 0.065 | 87 |
| SOPC:DOTAP | 5:5 | + | 0.076 | 102 |
| SOPC:DOPA | 9:1 | − | 0.078 | 105 |
| SOPC:DOPA:CHOL | 10:1:4 | — | 0.065 | 87 |

In table 1, SOPC stands for stearoyloleyl phosphatidylcholine, CHOL stands for cholesterol, DOTAP stands for dioleoyl trimethylammonium propane and DOPA stands for dioleoyl glycerophosphate.

In still another embodiment, the invention refers to a degradable oligomer or polymer hydrogel or microgel being positively or negatively charged and being further coated by means of one or more synthetic polyelectrolytes. Such a polyelectrolyte-coated degradable oligomer or polymer hydrogel or microgel may constitute a suitable swelling agent for entrapping a bio-agent or drug. Indeed, since said polyelectrolyte-coated gel has a core-shell structure wherein:

the core comprises a positively or negatively charged polymer hydrogel or microgel being able to entrap a drug, and

the shell comprises a synthetic polyelectrolyte and may serve as a semi-permeable membrane,

said coated is useful namely, but not only, for making a pulsed delivery system or time-controlled explosion system such as defined herein. This aspect of the invention is completely unexpected since polyelectrolyte shells are known in the art to be impermeable to molecules with a molecular weight higher than 5,000. To our knowledge, multi-layer coating was disclosed only with respect to decomposable colloidal particles for making capsules. For instance Shenoy et al. in Biomacromolecules (2003) 4:265-272 disclose polyelectrolyte layer-by-layer adsorption onto colloidal particles (having a size from 0.8 to 20 μm) of a biodegradable poly(DL-lactic-co-glycolic acid) hydrophobic copolymer and shows through SEM imaging that polyelectrolyte coating does not change the smooth surface morphology of particles. By contrast it was observed in this embodiment of the invention that polyelectrolyte coating of polymer hydrogels allows for a significant change in surface morphology by providing the coated hydrogel particles with a highly distinguishable brain-like aspect.

This embodiment of the invention is thus based on the degradation of a positively or negatively charged hydrogel or microgel having poly-electrolyte layers adsorbed on its surface. Hydrogels suitable for use in this embodiment of the invention include degradable oligomers and polymers as extensively described above and which may be positively or negatively charged through incorporation of an acidic monomer (e.g. acrylic acid or methacrylic acid) or a basic monomer (e.g. a dialkylaminoalkyl methacrylate, for instance wherein each alkyl group has from 1 to 3 carbon atoms) into their structure, but the invention is not limited to such illustrative examples. Methods for incorporating suitable amounts of such acidic or basic monomers into a degradable polymer of the type referred to herein are well known in the art.

This embodiment of the invention is also based on the interaction of a positively or negatively charged degradable hydrogel with one or more polyelectrolytes used as a coating. Suitable synthetic polyelectrolytes for this purpose include, but are not limited to, pH dependent cationic polyelectrolytes as well a pH independent and anionic polyelectrolytes. The term “pH dependent” as used herein means a weak electrolyte or polyelectrolyte, such as polyacrylic acid, in which the charge density can be adjusted by adjusting the pH. The term “pH independent” as used herein means a strong electrolyte or polyelectrolyte, such as polystyrene sulfonate, in which ionization is complete or nearly complete and does not change appreciably with pH. Other examples of suitable polyelectrolytes include poly(allylamine hydrochloride), sodium poly(styrene-sulfonate), polyacrylamide, polymethacrylic acid, poly(diallyldimethylammonium chloride), as well as biological polymers such as chitosan and dextran sulfate. Coating of a synthetic polyelectrolyte onto the surface of the charged hydrogel may be effected by any suitable technique known in the art, such as but not limited to the so-called layer-by-layer electrostatic self-assembly technique which is based on the alternate adsorption of oppositely charged polyelectrolytes on a charged surface, driven by the electrostatic interaction at each step of adsorption. The number of adsorption steps in this multi-step strategy is not particularly limited and may be from 2 to about 20, preferably from 3 to 10.

In a further improvement of this embodiment of the invention, hydrolysis of the polyelectrolyte-coated hydrogel may be accelerated, if needed for specific applications, by bringing said polyelectrolyte-coated hydrogel in contact with a suitable amount of an alkaline medium. For instance it was observed that, after submerging a polyelectrolyte-coated hydrogel of this invention in a 0.5 M NaOH solution, maximum swelling of the gel occurs within about 1 minute and, due to the increase in osmotic pressure caused by core degradation, stretching of the particle surface by a factor of about 4 reduces the shell thickness and increases permeability.

In the two latter embodiments of the invention (i.e. lipid coating and polyelectrolyte coating), the polymer hydrogel used may have an average size within a range from about 50 nm to about 10 μm, preferably from 1 to 5 μm, and a size distribution with a dispersity from about 1.1 to about 3.0, preferably from 1.2 to 2.0. The coating or multi-layer coating serving as an outer shell or semi-permeable membrane (being useful, among other applications, in a pulsed drug delivery system or time-controlled explosion drug release system) may suitably have a thickness within a range from about 10 nm to about 100 nm, preferably from 20 to 50 nm, as determined by standard analytical or imaging techniques well known in the art.

The term “bio-agent”, as used herein, is intended to mean any substance having biological activity such as, but not limited to, substances selected from the group consisting of therapeutic and prophylactic drugs and synthetic molecules, proteins, nucleic acids, vitamins, hormones, nutrients, aromas (fragances), fertilisers and pesticides, especially these where pulsed delivery is desirable for the biological activity involved.

The therapeutic agent may be selected for its specific properties such as for instance its anti-thrombotic, anti-inflammatory, anti-proliferative or anti-microbial efficiency. The latter include for instance anti-microbial agents such as broad spectrum antibiotics for combating clinical and sub-clinical infection, for example gentamycin, vancomycine and the like. Other suitable therapeutic agents are naturally occurring or synthetic organic or inorganic compounds well known in the art, including non-steroidal anti-inflammatory drugs, proteins and peptides (produced either by isolation from natural sources or recombinantly), hormones (for example androgenic, estrogenic and progestational hormones such as oestradiol, and gonadotropin releasing hormone for inducing fertility), bone repair promoters, carbohydrates, antineoplastic agents, antiangiogenic agents, vasoactive agents, anticoagulants, immunomodulators, cytotoxic agents, antiviral agents, antibodies, neurotransmitters, oligonucleotides, lipids, plasmids, DNA and the like. Suitable therapeutically active proteins include e.g. fibroblast growth factors, epidermal growth factors, platelet-derived growth factors, macrophage-derived growth factors such as granulocyte macrophage colony stimulating factors, ciliary neurotrophic factors, tissue plasminogen activator, B cell stimulating factors, cartilage induction factor, differentiating factors, growth hormone releasing factors, human growth hormone, hepatocyte growth factors, immunoglobulins, insulin-like growth factors, interleukins, cytokines, interferons, tumor necrosis factors, nerve growth factors, endothelial growth factors, osteogenic factor extract, T cell growth factors, tumor growth inhibitors, enzymes and the like, as well as fragments thereof. Suitable diagnostic agents include conventional imaging agents (for instance as used in tomography, fluoroscopy, magnetic resonance imaging and the like) such as chelates of a transition metal (e.g. a radioactive metal selected from the group consisting of ^{99m}Tc, ¹¹¹In, ⁶⁷Ga, ⁹⁰Y, ¹⁸⁶Re and ¹⁸⁸Re or a non-radioactive metal selected from gadolinium, manganese and iron).

Suitable anti-microbial agents include e.g. halogenated phenols, chlorinated diphenylethers, aldehydes, alcohols such as phenoxyethanol, carboxylic acids and their derivatives, organometallic compounds such as tributyltin compounds, iodine compounds, mono- and polyamines, sulfonium and phosphonium compounds; mercapto compounds as well as their alkaline, alkaline-earth and heavy metal salts; ureas such as trihalocarbanilide, isothia- and benzisothiazolone derivatives.

Suitable insecticides include natural ones, e.g. nicotine, rotenone, pyrethrum and the like, and synthetic ones like chlorinated hydrocarbons, organophosphorus compounds, biological insecticides (e.g. products derived from Bacillus thuringiensis), synthetic pyrethroids, organosilicon compounds, nitro-imines and nitromethylenes.

Suitable fungicides include e.g. dithiocarbamates, nitrophenol derivatives, heterocyclic compounds (including thiophtalimides, imidazoles, triazines, thiadiazoles, triazoles and the like), acylalanines, phenylbenzamides and tin compounds.

Suitable herbicides include e.g. trichloroacetic and aromatic carboxylic acids and their salts, substituted ureas and triazines, diphenyl ether derivatives, anilides, uraciles, nitriles and the like.

Suitable fertilisers include e.g. ammonium sulphate, ammonium nitrate, ammonium phosphate and the like, and mixtures thereof.

Therapeutic agents which are advantageously delivered according to the present invention belong to all permeability and solubility classes of the Biopharmaceutical Classification System according to G. Amidon et al. in Pharm. Res. (1995) 12:413-420. As will be appreciated by those skilled in the art, these drugs belong to various therapeutic classes including, but are not limited to, β-blockers, calcium antagonists, ACE inhibitors, sympathomimetic agents, hypoglycaemic agents, contraceptives, α-blockers, diuretics, anti-hypertensive agents, antipsoriatics, bronchodilators, corticosteroids, anti-mycotics, salicylates, cytostatics, antibiotics, virustatics, antihistamines, UV-absorbers, chemotherapeutics, antiseptics, estrogens, scar treatment agents, antifungals, antibacterials, antifolate agents, cardiovascular agents, nutritional agents, antispasmodics, analgesics and the like.

This invention is suitable e.g. for the following therapeutic or cosmetic agents: acebutolol, acetylcysteine, acetylsalicylic acid, acyclovir, alfuzosine, alprazolam, alfacalcidol, allantoin, allopurinol, alverine, ambroxol, amikacin, amiloride, aminoacetic acid, amiodarone, amitriptyline, amlodipine, amoxicillin, ampicillin, ascorbic acid, aspartame, astemizole, atenolol, beclomethasone, benserazide, benzalkonium hydrochloride, benzocaine, benzoic acid, betamethasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefatroxil, cefazolin, cefixime, cefotaxime, ceftazidime, ceftriaxone, cefuroxime, cephalosporins, cetirizine, chloramphenicol, chlordiazepoxide, chlorhexidine, chlorpheniramine, chlortalidone, choline, cyclosporin, cilastatin, cimetidine, ciprofloxacin, cisapride, cisplatin, clarithromycin, clavulanic acid, clomipramine, clonazepam, clonidine, clotrimazole, codeine, cholestyramine, cromoglycic acid, cyanocobalamin, cyproterone, desogestrel, dexamethasone, dexpanthenol, dextromethorphan, dextropropoxiphen, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavine mononucleotide, fluconazole, flunarizine, fluorouracil, fluoxetine, flurbiprofen, furosemide, gallopamil, gemfibrozil, Ginkgo biloba, glibenclamide, glipizide, clozapine, Glycyrrhiza glabra, griseofulvin, guaifenesin, haloperidol, heparin, hyaluronic acid, hydrochlorothiazide, hydrocodone, hydrocortisone, hydromorphone, ipratropium hydroxide, ibuprofen, imipenem, indomethacin, iohexol, iopamidol, isosorbide dinitrate, isosorbide mononitrate, isotretinoin, ketotifen, ketoconazole, ketoprofen, ketorolac, labetalol, lactulose, lecithin, levocarnitine, levodopa, levoglutamide, levonorgestrel, levothyroxine, lidocaine, lipase, imipramine, lisinopril, loperamide, lorazepam, lovastatin, medroxyprogesterone, menthol, methotrexate, methyldopa, methylphenidate, methylprednisolone, metoclopramide, metoprolol, miconazole, midazolam, minocycline, minoxidil, misoprostol, morphine, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, paroxetine, penicillins, phenobarbital, pentoxifylline, phenoxymethylpenicillin, phenylephrine, phenylpropanolamine, phenyloin, physostigmine, piroxicam, polymyxin B, povidone iodine, pravastatin, prazepam, prazosin, prednisolone, prednisone, bromocriptine, propafenone, propranolol, proxyphylline, pseudoephedrine, pyridoxine, quinidine, ramipril, ranitidine, reserpine, retinol, riboflavin, rifampicin, rutoside, saccharin, salbutamol, salcatonin, salicylic acid, simvastatin, somatotropin, sotalol, spironolactone, sucralfate, sulbactam, sulfamethoxazole, sulfasalazine, sulpiride, tamoxifen, tegafur, teprenone, terazosin, terbutaline, terfenadine, tetracaine, tetracycline, theophylline, thiamine, ticlopidine, timolol, tranexamic acid, tretinoin, triamcinolone acetonide, triamterene, triazolam, trimethoprim, troxerutin, uracil, valproic acid, verapamil, folinic acid, zidovudine, zopiclone, enantiomers thereof, organic and inorganic salts thereof, hydrates thereof and mixtures thereof, in particular mixtures in synergistic proportions.

Other bio-agents suitable for the purpose of the invention are vitamins, include those of the A group, of the B group (which means, besides B1, B2, B6 and B12, also compounds with vitamin B properties such as adenine, choline, pantothenic acid, biotin, adenylic acid, folic acid, orotic acid, pangamic acid, carnitine, p-aminobenzoic acid, myo-inositol and lipoic acid), vitamin C, vitamins of the D group, E group, F group, H group, I and J groups, K group and P group.

This invention is also suitable for therapeutic agents (drugs) having a water-solubility as low as about 0.2 μg/ml. Non-limiting examples of such drugs include for instance hydrochlorothiazide, nimodipine, flufenamic acid, mefenamic acid, bendroflumethiazide, benzthiazide, ethacrinic acid, nitrendipine and diamino-pyrimidines. Suitable examples of such poorly soluble diaminopyrimidines include, without limitation, 2,4-diamino-5-(3,4,5-trimethoxybenzyl) pyrimidine (trimethoprim), 2,4-diamino-5-(3,4-dimethoxy-benzyl) pyrimidine (diaveridine), 2,4 diamino-5-(3,4,6-trimethoxybenzyl) pyrimidine, 2,4-diamino-5-(2-methyl4,5-dimethoxybenzyl) pyrimidine (ormeto-prim), 2,4-diamino-5-(3,4-dimethoxy-5-bromobenzyl) pyrimidine, 2,4-diamino-5-(4-chloro-phenyl)-6-ethylpyrimidine (pyrimetha-mine), and analogues thereof.

This invention is suitable for said therapeutic agents (drugs) which further comprise one or more pharmaceutically acceptable excipients, such as emulsifiers or surface-active agents, thickening agents, gelling agents or other additives, and wherein the drug loading (i.e. the proportion of the drug in the formulation) may vary through a wide range from about 5% by weight to about 95% by weight.

Emulsifiers or surface-active agents suitable for therapeutic agents formulations include water-soluble natural soaps and water-soluble synthetic surface-active agents. Suitable soaps include alkaline or alkaline-earth metal salts, unsubstituted or substituted ammonium salts of higher, preferably saturated, fatty acids (C₁₀-C₂₂), e.g. the sodium or potassium salts of oleic or stearic acid, or of natural fatty acid mixtures obtainable form coconut oil, palm oil or tallow oil. Synthetic surface-active agents (surfactants) include anionic, cationic and non-ionic surfactants, e.g. sodium or calcium salts of polyacrylic acid; sulphonated benzimidazole derivatives preferably containing 8 to 22 carbon atoms; alkylarylsulphonates; and fatty sulphonates or sulphates, usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide) and the like. Suitable emulsifiers further include partial esters of fatty acids (e.g. lauric, palmitic, stearic or oleic) or hexitol anhydrides (e.g., hexitans and hexides) derived from sorbitol, such as commercially available polysorbates. Other emulsifiers which may be used include, but are not limited to, adducts of polyoxyethylene chains (1 to 40 moles ethylene oxide) with non-esterified hydroxyl groups of the above partial esters, such as the surfactant commercially available under the trade name Tween 60 from ICI Americas Inc.; and the poly(oxyethylene)/poly(oxypropylene) materials marketed by BASF under the trade name Pluronic.

Suitable structure-forming, thickening or gel-forming agents for the bio-agents of the invention include highly dispersed silicic acid, such as the product commercially available under the trade name Aerosil; bentonites; tetraalkyl ammonium salts of montmorillonites (e.g. products commercially available under the trade name Bentone) wherein each of the alkyl groups may contain from 1 to 20 carbon atoms; cetostearyl alcohol and modified castor oil products (e.g. a product commercially available under the trade name Antisettle). Gelling agents which may be included into the bio-agent formulation of the present invention include, but are not limited to, cellulose derivatives such as carboxymethylcellulose, cellulose acetate and the like; natural gums such as arabic gum, xanthum gum, tragacanth gum, guar gum and the like; gelatin; silicium dioxide; synthetic polymers such as carbomers, and mixtures thereof. Gelatin and modified celluloses represent a preferred class of gelling agents.

Other optional excipients which may be present in the bio-agent formulation of the present invention include additives such as magnesium oxide; azo dyes; organic and inorganic pigments such as titanium dioxide; UV-absorbers; stabilisers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavoring agents such as natural vanillin; buffers such as citric acid or acetic acid; extenders or bulking agents such as silicates, diatomaceous earth, magnesium oxide or aluminum oxide; densification agents such as magnesium salts; and mixtures thereof.

Time-controlled explosion bio-agent release systems and pulsed bio-agent delivery systems according to this invention may take different forms in terms of shape, size, composition and number of layers, including embodiments such as contemplated hereinbefore. For instance, the pulsed delivery system may be in the form of beads or granules comprising a core covered with one or more outer layers. They usually comprise at least (i) an outer semi-permeable membrane wherein bio-agent release or delivery is caused by explosion of the said membrane and begins after a certain lag time and at least (ii) a core comprising a bio-agent and a swelling agent, and are further characterised in that the said swelling agent is a degradable polymer aqueous solution or hydrogel, preferably of the type wherein degradation occurs by cleavage of the polymer backbone or by cleavage of cross-linking bonds within the hydrogel. Although a membrane and a core such as above defined are the main requirements of such systems, more elaborate structures such as including additional intermediate layers comprising further excipients (such as defined hereinbefore) cannot be excluded.

In a suitable working embodiment of the invention, the bio-agent is present in the core in the form of micro- or nanoparticles. The bio-agent may also be intimately admixed with the swelling agent.

The present invention is not limited to releasing the bio-agent as a single pulse. In specific cases, it may be beneficial to provide multiple pulsed delivery or multiple explosion release of the bio-agent. This may be effected by providing the delivery system, e.g. the core of said delivery system, with a mixture of at least two swelling agents having different degradation rates such as to provide two or more different lag times for the bio-agent(s). In a specific embodiment for this purpose, the core of the delivery system may comprise at least a first population of micro- or nanoparticles including a first bio-agent and a first swelling agent and a second population of micro- or nanoparticles including a second bio-agent and a second swelling agent, so that the first bio-agent is released or delivered after a first lag time and the second bio-agent is released or delivered after a second lag time, the said second lag time being substantially different from the said first lag time. In this embodiment, the first bio-agent may be different from or the same as the second bio-agent, thus providing additional flexibility for the biological, e.g. therapeutic or prophylactic, treatment. As previously mentioned, each of the first and subsequent lag times may independently vary within very broad ranges from about one hour to two weeks.

The pulsed delivery or explosion release systems of the present invention are suitable for a number of different ways of administration of therapeutic agents such as, but not limited to, oral administration, parenteral administration, subcutaneous administration, vaccination and the like.

In another aspect, the invention relates to a method of protecting plants or crops by releasing a bio-agent selected from the group consisting of fertilisers, anti-microbial agents, insecticides, fungicides, herbicides and pesticides onto said plants or crops, wherein said bio-agent is included in a time-controlled explosion bio-agent release system or pulsed bio-agent delivery system comprising at least (i) an outer semi-permeable membrane wherein bio-agent release or delivery is caused by explosion of said semi-permeable membrane and begins after a lag time and at least (ii) a core comprising a bio-agent and a swelling agent, wherein said swelling agent is a degradable oligomer or polymer aqueous solution or hydrogel wherein degradation occurs by cleavage of the polymer backbone and/or, in the case of a hydrogel, by cleavage of cross-linking bonds within said hydrogel. Said method preferably comprises spraying time-controlled explosion bio-agent release system or pulsed bio-agent delivery system onto the plants or crops to be protected, either in solid form or, more preferably for dose control, as a dispersion in a suitable liquid medium.

Although the swelling behaviour of polymer networks has been the subject of numerous investigations, according to our knowledge the variation of the swelling pressure in degrading gel systems has not previously been the subject of significant investigations. Therefore it should be understood that this invention may be performed in a number of different ways without departing from its original concept. In particular, it was observed that the release of degradation products and the swelling pressure profile may be strongly dependent upon the degradation mechanisms of the polymers involved. More especially, at least for modified dextran gels, the swelling pressure increases rather continuously when degradation occurs at their backbone, whereas swelling pressure increases more discontinuously (with a final sudden increase) when degradation occurs at their crosslinks. Therefore it is possible to take advantage of this discriminating behaviour by tailoring the composition of the degradable polymer involved in the invention in view of the desired release profile of the bio-active composition.

The following set of examples provides a selection of a few appropriate working embodiments for this invention which should be understood as purely illustrative and without any limiting intention.

### EXAMPLE 1

### Dex-HEMA Preparation and Characterization

Dex-HEMA batches were prepared and characterized according to the method described by Van Dijk et al. (cited supra), using dextran (commercially available from Fluka, obtained from Leuconostoc ssp.) with a molecular weight Mₙ=19 000. The degree of substitution (hereinafter DS) of Dex-HEMA was determined by proton nuclear magnetic resonance spectroscopy (H-NMR) in D20 with a Gemini 300 spectrometer (Varian). The DS of samples used in the following examples were 2.9, 5.0 and 7.5, respectively.

### EXAMPLE 2

### Preparation and Degradation of Dex-HEMA Hydrogels

Dex-HEMA gels were made by radical polymerization of aqueous dex-HEMA solutions by first dissolving dex-HEMA in a phosphate buffer (10 mM Na₂HPO₄, 0.02% sodium azide, adjusted with 1 N hydrochloric acid to pH 7.0). The polymerization reagents were TEMED (50 μl of a 20% volume/volume solution in deoxygenated phosphate buffer, pH 8.5, added to 1 g polymer solution) and KPS (90 μl of a 50 mg/ml solution in deoxygenated phosphate buffer). The reactor was coated with polyethylene glycol (hereinafter PEG) (M_{w} 20,000; 10% solution in phosphate buffer) in order to reduce adhesion. Gelation required about 1 hour at 23° C. Hydrogel samples for the following rheology measurements were made in cylindrical molds (diameter 23 mm, height 2 mm).

For the other experiments, gels were prepared in 2.5 ml polypropylene syringes (diameter 8.5 mm) from which the heads were sawn. After gelation the gel samples were removed from the syringe and cut with a thin wire. Degradation was studied in phosphate buffer (pH 7) at 37° C. Throughout the following examples, the dex-HEMA concentration (expressed in weight %) refers to the concentration at which cross-links were introduced.

### EXAMPLE 3

### Osmotic De-Swelling of Dex-HEMA Hydrogels

Osmotic deswelling measurements were performed on dex-HEMA gels using the method described by Horkay et al. in Macromolecules (1982) 15:1306-1310. Gel specimens were surrounded by a semi-permeable membrane (Medicell dialysis bags, M_{w} between 12 000 and 14 000). Similar dialysis bags were used in the purification step of the synthesis of dex-HEMA in example 1 above.

After different degradation times, gel samples were equilibrated with PEG-solutions at 4° C. PEG (available from Merck, M_{w}=20,000) was dissolved in citrate buffer (9.44 g/l Na₂HPO₄; 10.3 g/l citric acid and 0.2 g/l NaN₃, pH 4.4). The PEG concentration was varied in the range from 0 to 12.5 g/100 ml. It was verified that further degradation of the dex-HEMA gels did not occur during the osmotic de-swelling measurements. Equilibrium swelling was attained within 7 days. The reversibility of the swelling process was checked.

At equilibrium, the swelling pressure of the gel is equal to the osmotic pressure of the PEG-solution. The osmotic pressure of the PEG-solution was calculated from the equation disclosed by Nichol et al. in Biochem J. (1967) 102:407-416 as follows:

$${Π}_{PEG} = [ \frac{1}{{M}_{n}} + {A}_{2} c + {A}_{3} {c}^{2} ] \cdot cRT \times 10 \qquad ( 1 )$$

where R is the gas constant, T is the absolute temperature, c is the PEG concentration (in g/100 ml), and A₂ and A₃ are the second and third virial coefficient, respectively. According to the data reported by Edmond et al. in Biochem J. (1968) 109:569-576 for PEG (Mₙ=20,000), A₂=2.59×10⁻⁵ (mol. 10² ml)/g² and A₃=1.35×10⁻⁶ (mol. 10⁴ ml²)/g³.

The dex-HEMA concentration of the gels was calculated using the relationship

$$c = \frac{{w}_{dex \text{-} HEMA}}{[ ( {w}_{dex \text{-} HEMA} \times {v}_{1} ) + ( \frac{{w}_{e} - {w}_{dex \text{-} HEMA}}{ρ} ) ]} \times 100 \qquad ( 2 )$$

where wₑ is the weight of the dex-HEMA gel, W_{dex-HEMA} is the weight of dex-HEMA determined gravimetrically after drying the gel in a vacuum oven at 50° C., ρ is the density of the buffer and v₁ is the specific volume of the dex-HEMA (v₁=0.72 ml/g) as disclosed by De Smedt et al. in Macromolecules (1995) 28:5082-5088. The polymer volume fraction (φ) of the gels was calculated from the concentration of dex-HEMA and v₁.

### EXAMPLE 4

### Mechanical Characterization of Degrading Dex-HEMA Hydrogels

Rheological measurements were performed using an AR1000-N controlled stress rheometer (available from TA-Instruments). In order to avoid slippage, the acrylic top plate was covered by sandpaper (diameter 2 cm). The bottom plate was replaced with a Plexiglas® plate with a roughened surface. Measurements were done in oscillation mode at 1 Hz in the linear visco-elastic region of these gels by applying a constant strain of 0.5%. After measurement, the hydrogel samples were transferred into phosphate buffer and stored at 37° C. Further details of this method are provided by Meyvis et al. in J. Rheol. (1999) 43:933-950.

### EXAMPLE 5

### Determination of Free Dextran Chains in Dex-HEMA Gels

The concentration of free dextran in the dex-HEMA hydrogels of example 2 was determined from a release experiment performed in phosphate buffer at 37° C. The amount of dextran chains in the solution was measured by gel permeation chromatography (GPC) in a system consisting of a high pressure pump (Waters M510), an injector (Waters U6K) and a differential refractometer (Waters 410). 250 μl of each sample was injected and a flow rate of 0.5 ml/min was applied. The dex-HEMA concentration was calculated from the height of the peak using a calibration curve (between 0 and 2.5 mg/ml) obtained for the corresponding dex-HEMA.

Results and Discussion

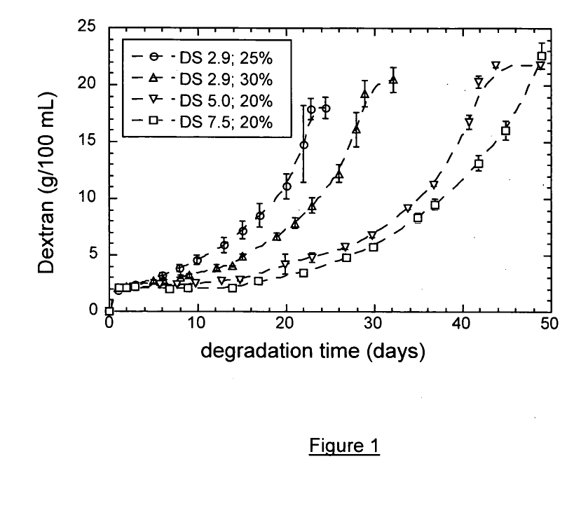

FIG. 1 shows the amount of dextran released from different dex-HEMA gels as a function of degradation time: first the sol fraction (unreacted dex-HEMA chains) leaves the gel, this feature being independent of the degradation process. In the second region (delay region) a relatively small amount of dextran is released. Finally, when the majority of cross-links are cleaved, liberation of dextran chains is significantly enhanced.

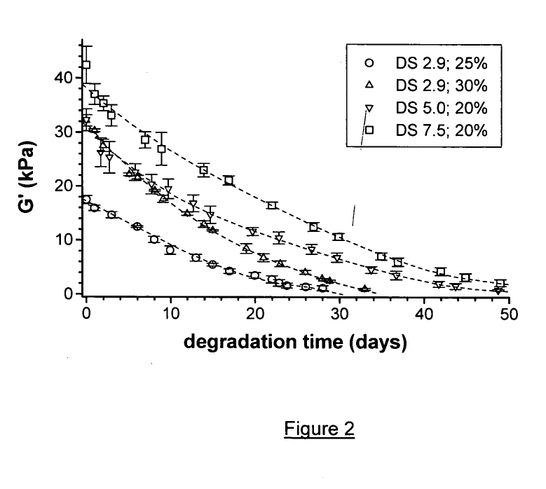

FIG. 2 shows the elastic modulus G′ as a function of the degradation time and exhibits a continuous decrease during the degradation process. The decrease of G′ is significantly slower in gels having higher dex-HEMA concentration or higher DS. Since G′ is proportional to the cross-link density, this finding indicates that degradation is slower in densely cross-linked gels.

In order to elucidate the effect of degradation on thermodynamic properties we measured the swelling pressure π_{sw} at different stages of degradation. The swelling pressure π_{sw} of a non-ionic gel can be described as the sum an osmotic pressure πₒₛₘ that expands the network and an elastic pressure πₑₗ that acts against expansion:

π_{sw}=πₒₛₘ+πₑₗ (3)

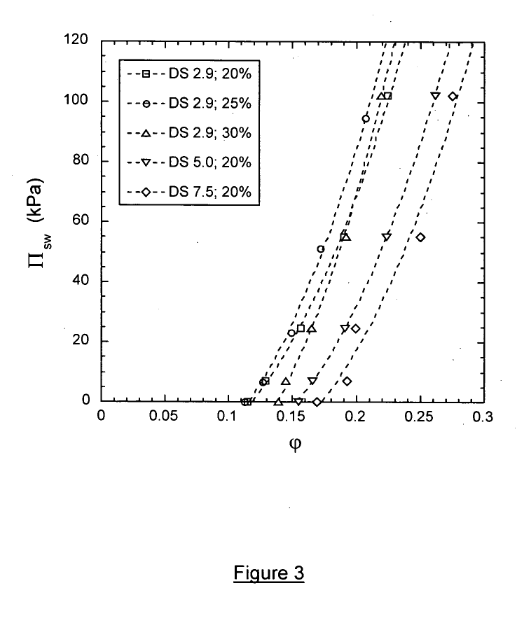

FIG. 3 shows the swelling pressure as a function of the polymer volume fraction for different undegraded dex-HEMA hydrogels. The continuous curves are the least squares fits of the swelling pressure data according to Horkay et al. (citedsupra):

π_{sw}=Aφⁿ−Aφₑ^{n-1/3}φ^{1/3} (4)

where A is a constant depending on the particular polymer-solvent system, φₑ and φ are the volume fraction of the polymer in equilibrium with pure buffer and PEG solutions, respectively. For the exponent n, the scaling theory (P. G. De Gennes, Scaling concept in polymer physics, published 1979) predicts n=2.31 (good solvent condition) and n=3.0 (Θ-solvent condition). The values of A and n obtained from the fits to equation 4 are listed in Table 1 hereinafter. As expected A depends on the chemical composition of the network. The value of n is close to that predicted for good solvent condition.

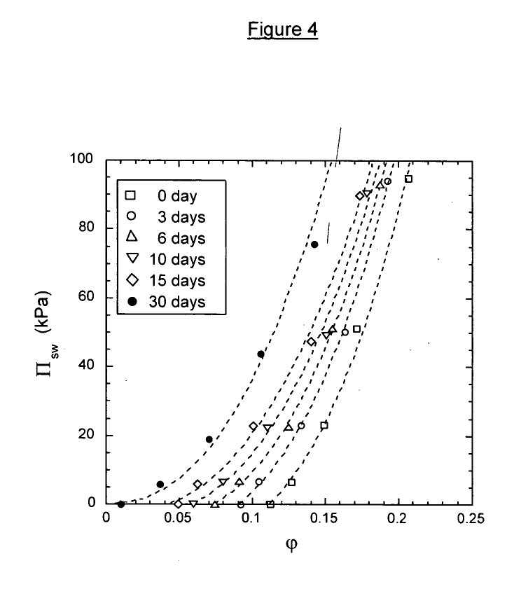

The effect of degradation on the swelling pressure was studied on the sample having the shortest degradation time (Dex-HEMA DS2.9; 25%). FIG. 4 shows the swelling pressure as a function of the polymer volume fraction measured at different stages of degradation (up to 30 days). The π_{sw} versus φ curves are gradually shifted to the left as the gel degrades.

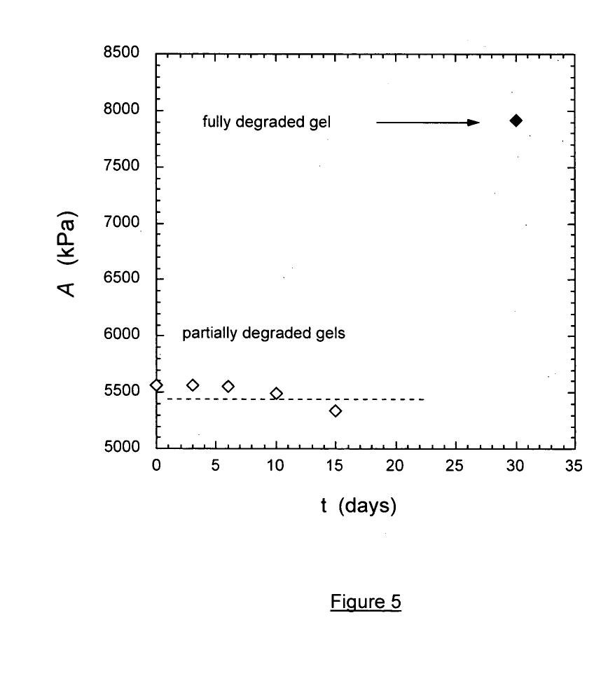

The parameters obtained from the fits to equation 4 at different degradation times (table 2 and FIG. 5) indicate that neither A or n varies noticeably during the first 15 days of degradation. It can also be seen that after 30 days (i.e., when the network became completely liquid) the value of A significantly increases.

The dashed line in FIG. 4 shows the situation that occurs when the gel is surrounded by a rigid semi-permeable membrane. During degradation, π_{sw} increases from 0 kPa (swelling pressure of the fully swollen non degraded gel) to 49 kPa (swelling pressure of the totally degraded dex-HEMA gel). The latter is the hydrostatic pressure required to maintain the initial concentration (φ=0.112) of the gel during the degradation process.

The above results indicate that the degradation rate strongly depends on the initial dex-HEMA concentration and DS. The variation of the swelling pressure at each stage of degradation is satisfactorily described by equation (4). In the earlier phase of the degradation process, the swelling pressure gradually increases because of the decrease of the elastic pressure. Towards the end of the degradation process, a pronounced increase in the swelling pressure is observed and is accompanied by a sudden increase in the amount of dextran released from the gel.

In drug delivery systems, osmotic pressure can cause rupture of the membrane surrounding the hydrogel particle. Consequently, the detailed knowledge of the variation of the swelling pressure during the degradation process, such as investigated by the above methodology, is essential to design systems based on degradable hydrogels that have a swelling pressure profile tailored for pulsed delivery of drugs.

TABLE 2

| | degradation time | volume fraction | A | |
|---|---|---|---|---|
| sample | (days) | ψ^{a} | (kPa) | n |
| DS 7.5; 20% | 0 | 0.1150 | 4554 | 2.35 |
| DS 5.0; 20% | 0 | 0.1391 | 5767 | 2.33 |
| DS 2.9; 20% | 0 | 0.1547 | 3587 | 2.33 |
| DS 2.9; 30% | 0 | 0.1688 | 3231 | 2.36 |
| DS 2.9; 25% | 0 | 0.1128 | 5562 | 2.36 |
| DS 2.9; 25% | 3 | 0.0919 | 5563 | 2.34 |
| DS 2.9; 25% | 6 | 0.0745 | 5554 | 2.35 |
| DS 2.9; 25% | 10 | 0.0598 | 5487 | 2.34 |
| DS 2.9; 25% | 15 | 0.0493 | 5337 | 2.30 |
| DS 2.9; 25% | 30 | <0.01 | 7920 | 2.33 |

^{a}volume fraction at equilibrium swelling

### EXAMPLE 6

Dex-HEMA/dextran hydrogels were made as in example 2. Solutions were prepared by dissolving dex-HEMA and dextran in phosphate buffer. The same dextran (from Leuconostoc mesenteroides, Merck, Mₙ=19,000) was used as in dex-HEMA synthesis.

The swelling pressure of gels prepared in the presence of free dextran chains was determined by a swelling pressure osmometer consisting of a calibrated transducer (Honeywell), a sample chamber (volume 4.2 mL) and a buffer chamber (filled with 15 mL phosphate buffer at pH 7.0); the chambers are separated by a semi-permeable membrane (Medicell, M_{w} cut-off between 12,000 and 14,000) supported by a porous Bekipor® frame which is further supported by a Teflon perforated cylinder. The membrane is permeable to small molecules (water and ions) but impermeable to large dextran molecules. The apparatus measures π_{sw} up to 7 atmospheres. π_{sw} measurements were performed on gels made in the sample chamber 12 hours after prepration, i.e. before substantial degradation occurred. Measurements were made at 4° C., thus preventing hydrolysis of the dex-HEMA/dextran hydrogels. The reproducibility of the swelling pressure measurements was found to be better than ±2%.

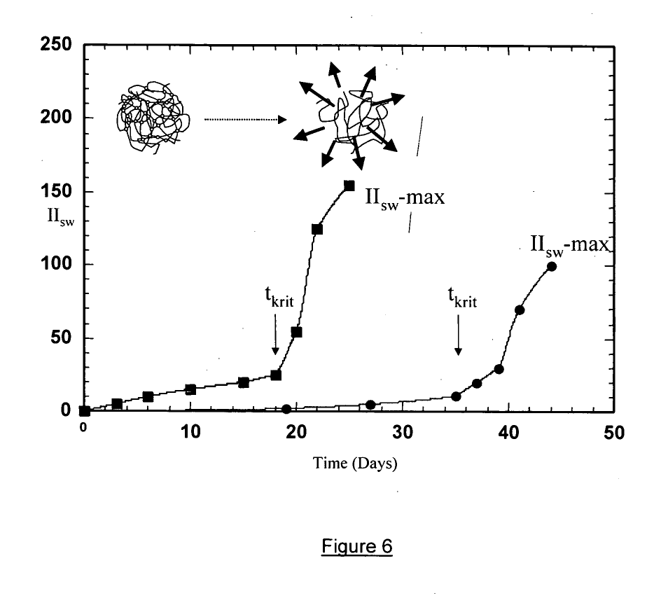

In FIG. 6 are presented swelling pressure data (π_{sw}, expressed in kPa) versus degradation time plots obtained from swelling pressure measurements of:

a dex-HEMA/dextran gel with a DS of 2.9, at a concentration of 25% by weight (left, showing a critical time of about 18 days), and

a dex-HEMA/dextran gel with a DS of 5.0, at a concentration of 20% by weight (right, showing a critical time of about 36 days).

### EXAMPLE 7

### Swelling Pressure of Dex-MA Hydrogels

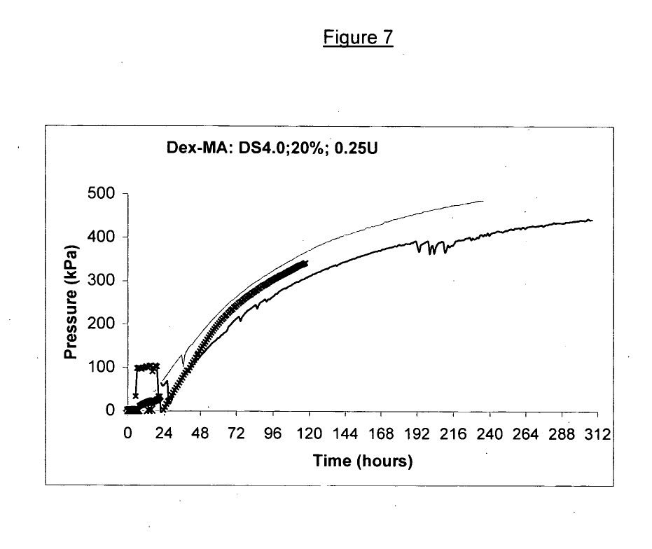

Methacrylated dextran (Dex-MA) with a DS=4.0 was prepared according to the method disclosed by Van Dijk et al. in Macromolecules (1995) 28:6317-6322. Hydrogels were prepared by radical polymerisation of an aqueous solution of dex-MA, said solutions being prepared by dissolving the dex-MA thus obtained in phosphate buffer (PB) (10 mM Na₂HPO₄, 0.02% sodium azide, adjusted with 1 N hydrochloric acid to pH 7.0) at a concentration of 20% by weight. Prior to addition of the gelation reagents, the enzyme solution (D-1508 Sigma; diluted to 10 U/ml in 10 mM PB pH 7.0; one unit delivers 1 μmole of isomaltose per minute at pH 6 at 37° C.) was added to the dex-MA solution (cooled to 4° C.) in such a way that its final concentration corresponds to 0.25 unit per gram of gel. Gelation started after adding 50 μl TEMED (commercially available from Fluka; 20% by volume in deoxygenated phosphate buffer, pH adjusted to 8.5 with hydrochloric acid) per gram, followed under stirring by 90 μl KPS (commercially available from Fluka; 50 mg/ml in deoxygenated phosphate buffer) per gram. Gels were immersed directly into the membrane osmometer of example 6 for determination of the swelling pressure of the enzymatically degrading Dex-MA hydrogels. The data of swelling pressure measurements are shown in FIG. 7.

### EXAMPLE 8

### Osmotic Pressure Changes of Lactic Acid-Polyethylene Glycol Block Copolymers

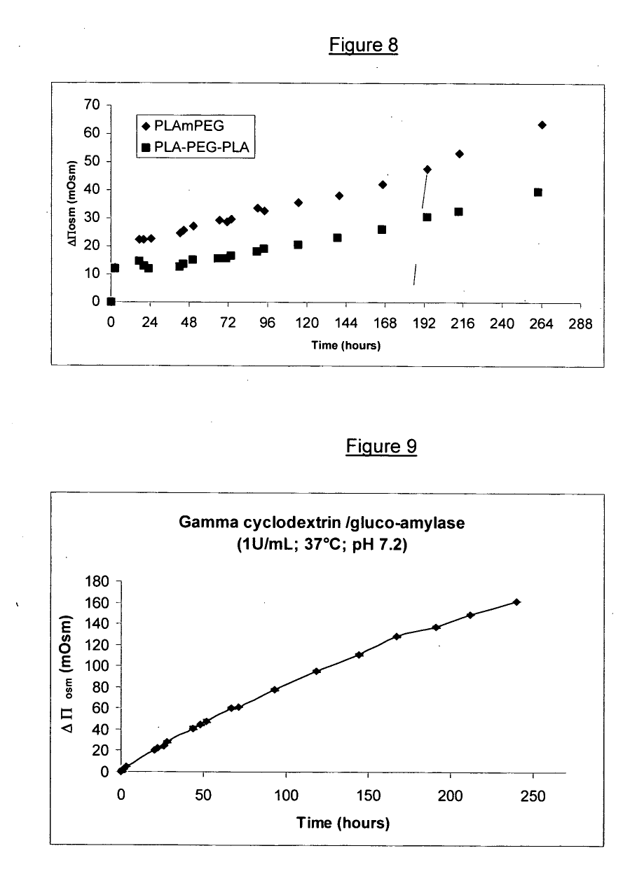

The change in osmotic pressure of two different degrading polymer solutions was measured by using freezing—point osmometry (using the Advanced Micro-osmometer Model 3300, commercially available from Advanced Instruments, Inc.). Chemical hydrolysis of a (lactic acid-b-polyethylene glycol) diblock copolymer (with block molecular weights of 456 and 2,000 respectively) and a (lactic acid-b-polyethylene glycol-b-lactic acid) triblock copolymer (with block molecular weights of 231, 1450 and 231 respectively) was followed as a function of time. Therefore 5% by weight solutions were made of each polymer in N-2-hydroxyethylpiperazine-N′-2-ethanesulfonic acid (HEPES) buffer (50 mM, pH 7.2). Both copolymer solutions were degraded at 37° C. 20 μL samples were taken at well defined times and the osmotic pressure (in mOsm) was measured immediately. Measurement results are shown in FIG. 8.

### EXAMPLE 9

### Osmotic Pressure Changes of γ-Cyclodextrin

The change in osmotic pressure of a γ-cyclodextrin solution was measured as in example 8. Enzymatic hydrolysis of γ-cyclodextrin with muco-amylase (0.06 mg/mL, corresponding to 1 U/mL) was followed as a function of time. Therefore a 5% by weight solution of γ-cyclodextrin was made in HEPES-buffer (50 mM, pH of 7.2) and then degraded at 37° C. 20 μL samples were taken at well defined times and the osmotic pressure (in mOsm) was measured immediately. Measurement results are shown in FIG. 9.

### EXAMPLE 10

### Preparation of Dex-HEMA Microgels

Dex-HEMA microgels were prepared as follows. Deoxygenated aqueous solutions of dex-HEMA (25% w/w solution) and PEG (24% by weight solution; M_{w} 20,000) were prepared. Dex-HEMA and PEG solutions (in a PEG/dex-HEMA volume ratio of 40:1) were vigorously mixed with a vortex for 1 minute under a nitrogen atmosphere in order to obtain 5 mL of a water-in-water emulsion. This emulsion was allowed to stabilize for 15 minutes. Subsequently TEMED (0.100 μl; pH neutralized with 4 N HCl) and KPS (180 μl; 41 mM) were added for cross-linking dex-HEMA. After gentle mixing the emulsion was incubated without stirring for 30 minutes at 25° C., thus yielding microgels with an estimated water content of 75% by weight. Residual KPS and TEMED were removed by three washing and centrifugation steps with 50 mL Milli-Q water. The remaining pellets were suspended in 5 mL phosphate buffer (10 mM at pH of 7.0).

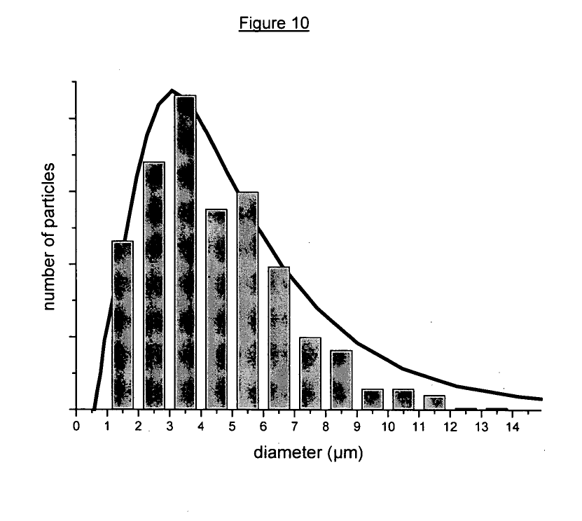

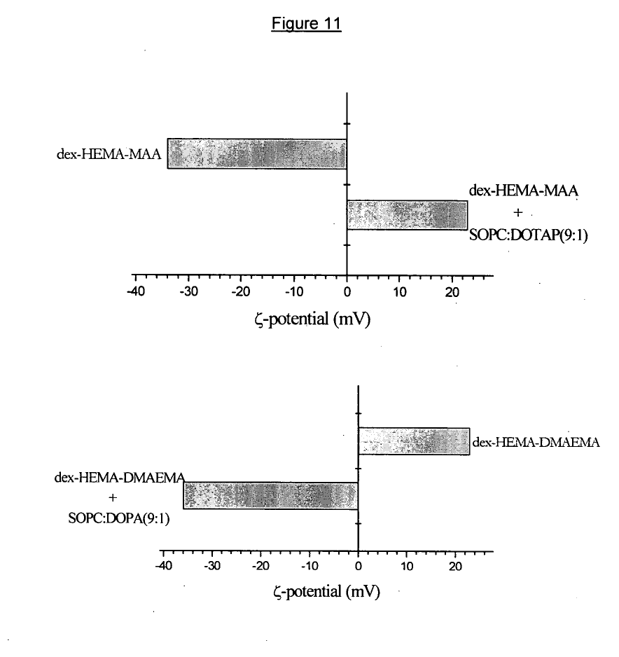

In order to prepare respectively negatively and positively charged dex-HEMA microgels, respectively methacrylic acid (MAA; 25 μl) or dimethyl aminoethyl methacrylate (DMAEMA; 35 μl) was added to the PEG/dex-HEMA mixture described above prior to vortexing. In order to prepare fluorescent microgels, 4 mg/mL tetramethyl rhodamine B isothiocyanate (TRITC) labeled dextran (M_{w} of 158,000) was added to the dex-HEMA solution used in the preparation of the microgels. Size distribution of the dex-HEMA microgels was characterized by transmission light microscopy and laser diffraction, results being shown in FIG. 10. A number average diameter of about 3 μm was obtained by both methods, with a rather broad size distribution (mainly from 1 to 7 μm) due to the water-in-water emulsion technique.

### EXAMPLE 11

### Preparation of Lipid Vesicles

Lipid vesicles (liposomes) were prepared as follows. First lipids were dissolved in chloroform, then chloroform was evaporated at room temperature using nitrogen and the lipid film was further dried under vacuum for 12 hours in order to remove any remaining chloroform. Large multi-lamelar vesicles were obtained by hydration of the dry lipid film with a carboxyfluorescein (hereinafter referred as CF) solution (100 mM CF, 0.95 M NaCl in 50 mM HEPES at a pH of 7.4; 2180 milliosmole (mOsm)) up to a final lipid concentration of 5 mg/mL. Uni-lamelar vesicles were then obtained by extruding the sample eleven times through two stacked polycarbonate filters (100 nm pore size, available from Nucleopore) using an extruder (Avanti Polar Lipids). Vesicle size distribution was determined by dynamic light scattering (using an Autosizer 4700 equipment from Malvern Instruments).

The following lipid compositions were used

(i) dioleoyl phosphatidylcholine (DOPC) and cholesterol (CHOL) in DOPC:CHOL molar ratios of 10:0, 9:1, 7:3 and 5:5 respectively;

(ii) stearoyloleyl phosphatidylcholine (SOPC) and dioleoyl trimethylammonium propane (DOTAP) in SOPC:DOTAP molar ratios of 9:1, 7:3 and 5:5 respectively;

(iii) SOPC and dioleoyl glycerophosphate (DOPA) in a SOPC:DOPA molar ratio of 9:1; and

(iv) A mixture of SOPC, DOPA and CHOL in a SOPC:DOPA:CHOL molar ratio of 4:1:5.

All lipids were available from Avanti Polar Lipids. After extrusion, unentrapped CF was removed by passing the sample down a Sephadex G-50 column (1.5×10 cm) equilibrated with a solution with the same osmotic activity as the CF solution inside the lipid vesicles (i.e. 2,180 mOsm).

### EXAMPLE 12

### Lipid Coating of Dex-HEMA Microgels

The lipid film prepared as described in example 11 was hydrated by adding Milli-Q water (until a final lipid concentration of 1 mg/mL) and sonicated (using a Bransonic 32 equipment from Branson Ultrasonics, 150 watts) for 5 minutes. A small amount (0.05 mole % of the total lipid) of the lipid soluble fluorescent dye cholesteryl BODIPY-FL C12 (λex 504 nm; λem 511 nm; available from Molecular Probes) was added to make the lipid coating fluorescent.

The charged lipid vesicles (500 μL) were mixed with a suspension (200 μL) of the oppositely charged microgels prepared according to example 10 and incubated for 20 minutes to allow adsorption of the lipid vesicles to the surface of the microgels. Then the samples were centrifuged three times (using a Microfuge 18 Centrifuge equiment from Coulter Beckman) for 5 minutes at 500 g and the supernatant was removed.

### EXAMPLE 13

### Electrophoretic Mobility of Lipid Coated Microgels

The electrophoretic mobility of the lipid coated microgels obtained in example 12 was measured by means of a Malvern Zetasizer 2000 (available from Malvern Instruments) and compared to that of microgels obtained in example 10. The dex-HEMA microgel dispersion was centrifuged for 1 minute at low speed (500 rpm) and measurements were done on microgels that remained in the supernatant. The ζ-potential (expressed in mV) was calculated from the electro-phoretic mobility by using the Smoluchowski relation both for uncoated microgels and lipid coated microgels. Results are shown in FIG. 11, wherein the upper part of the figure relates to negatively charged dex-HEMA-MM microgels coated with the positively charged lipid obtained from a SOPC/DOTAP mixture in a 9:1 ratio, and wherein the lower part of the figure relates to positively charged dex-HEMA-DMAEMA microgels coated with the negatively charged lipid obtained from a SOPC/DOPA mixture in a 9:1 ratio. FIG. 11 clearly shows that the zeta-potential of negatively and positively charged dex-HEMA microgels turns respectively positive and negative upon exposing them to the oppositely charged lipid vesicles of example 11.

### EXAMPLE 14

### Layer-by-Layer (LbL) Coating of Dex-HEMA Microgels

Dex-HEMA microgels obtained in example 10 were coated by the consecutive adsorption of oppositely charged polyelectrolytes using the following centrifugation technique. The microgels (50 mg) were dispersed in 1 mL of a polyelectrolyte solution (2 mg/mL in 0.5M NaCl, except for chitosan 1 mg/mL in 0.5 M NaCl). The polyelectrolytes were allowed to adsorb for 15 minutes, under continuous gentle shaking. The dispersion was then centrifuged at a speed of 3000 rpm for 3 minutes. Subsequently the supernatant was removed and the microgels were redispersed in Milli-Q water to wash away the non-adsorbed polyelectrolytes. This washing was repeated twice before the second polyelectrolyte solution was added. The process was repeated 3 times until the desired LbL coating was reached. Polyelectrolytes used in this example include chitosan (a cationic polymer with high molecular weight), sodium poly(styrenesulfonate) (PSS, M_{w}˜70,000), poly(allylamine hydrochloride) (PAH, M_{w}˜70,000) and poly(diallyl dimethyl ammonium chloride) (PDADMAC, M_{w}˜100,000-200,000), all being obtained from Aldrich.

### EXAMPLE 15

### Electrophoretic Mobility of Layer-by-Layer (LbL) Coated Dex-HEMA Microgels

The electrophoretic mobility of the layer-by-layer coated microgels obtained in example 14 was measured by using the same methodology and equipment as in example 13.

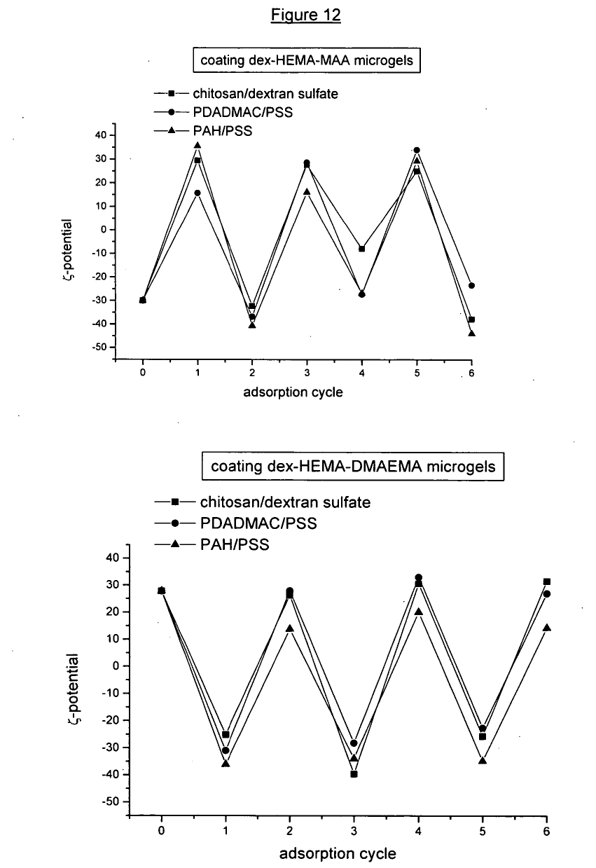

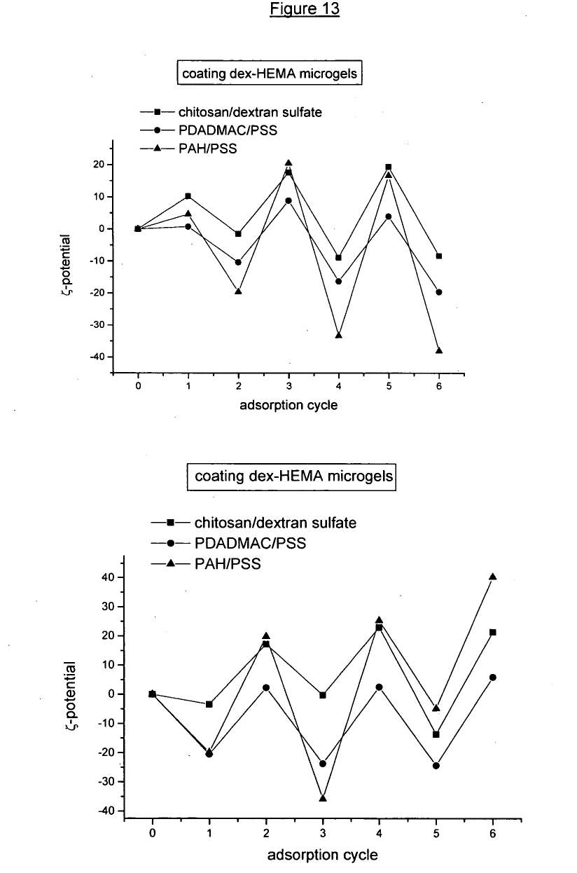

FIGS. 12 and 13 show the results of ζ-potential measurements on uncoated and LbL coated dex-HEMA microgels respectively. Before coating, the ζ-potentials of neutral, dex-HEMA-MM and dex-HEMA-DMAEMA microgels were respectively 0 mV, −30 mV and 27.8 mV. Figures clearly shows that the charge of the microgels changes upon submerging them in the polyelectrolyte solution, indicating that multilayer build-up takes place. FIGS. 12 and 13 also clearly indicate that the microgels of example 10 can be coated with the polyelectrolyte combination of dextran sulfate and chitosan, likely due to the porous and hydrophilic nature of these microgels, leading to high interpenetration with previously adsorbed layers and loops extending into the solution carrying the excess charge.

FIGS. 12 and 13 show that LbL coating of the neutral dex-HEMA microgels is also possible. For charged microgels (FIG. 12), electrostatic interactions between the gel and the polyelectrolytes are the main driving force for polyelectrolyte adsorption. These interactions are strong enough to avoid that the adsorbed layers are removed upon adsorption of the next polyelectrolyte layer.

### EXAMPLE 16

### Microscopic Imaging of Layer-by-Layer (LbL) Coated Dex-HEMA Microgels

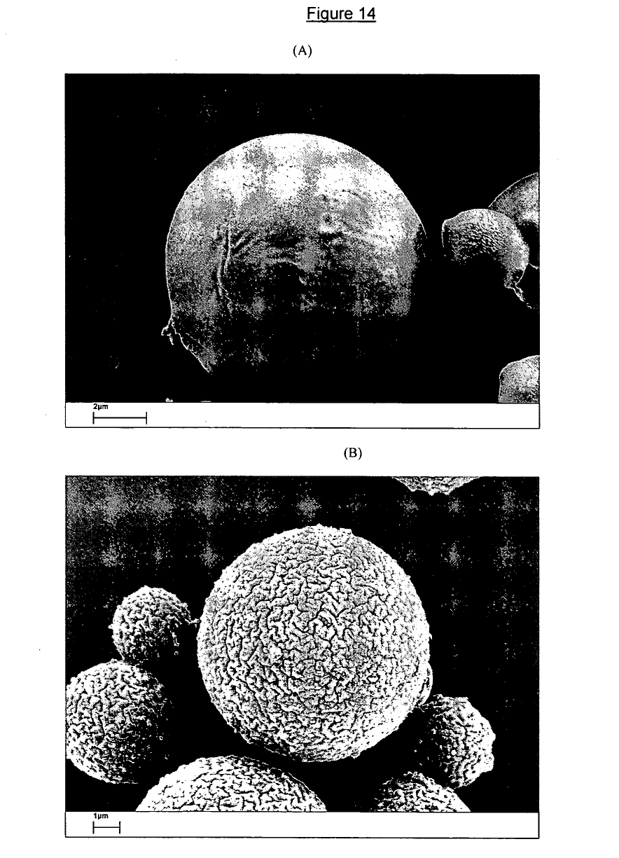

In order to get information about the morphology of the LbL coated microgels obtained in example 14, scanning electron microscopy (SEM) images were taken using a Zeiss DSM 40 instrument (Zeiss, Germany) operating at an accelerating voltage of 3 keV. FIG. 14 shows SEM images of both uncoated (upper part of the figure) and microgels coated with 3 PSS/PAH bilayers (lower part of the figure). It reveals that the surface of uncoated microgels is rather smooth when compared to the coated ones which show a remarkably more granular “brain-like” structure.

1. A time-controlled explosion bio-agent release system or pulsed bio-agent delivery system comprising at least (i) an outer semi-permeable membrane wherein bio-agent release or delivery is caused by explosion of said semi-permeable membrane and begins after a lag time and at least (ii) a core comprising a bio-agent and a swelling agent, wherein said swelling agent is a degradable oligomer or polymer aqueous solution or hydrogel wherein degradation occurs by cleavage of the polymer backbone and/or, in the case of a hydrogel, by cleavage of cross-linking bonds within said hydrogel.

2. A time-controlled explosion release system or pulsed delivery system according to claim 1, wherein the lag time is at least partially controlled by the degradation rate of said degradable oligomer or polymer aqueous solution or hydrogel.

3. A time-controlled explosion release system or pulsed delivery system according to claim 1, wherein said degradable oligomer contained in said aqueous solution is selected from the group consisting of disaccharides, oligosaccharides and polysaccharides.

4. A time-controlled explosion release system or pulsed delivery system according to claim 1, wherein said degradable oligomer or polymer has a molecular weight ranging from about 100 to about 1,000,000.

5. A time-controlled explosion release system or pulsed delivery system according to claim 1, wherein said degradable hydrogel is a degradable modified dextran.

6. A time-controlled explosion release system or pulsed delivery system according to claim 1, wherein said degradable hydrogel is a dextran modified by means of at least one (C₁₋₈ alkyl) acrylate or methacrylate and wherein degradation occurs by cleavage of the polymer backbone.

7. A time-controlled explosion release system or pulsed delivery system according to claim 1, wherein said degradable hydrogel is a dextran modified by means of at least one (hydroxy C₁₋₈ alkyl) acrylate or methacrylate and wherein degradation occurs by cleavage of cross-linking bonds within said hydrogel.

8. A time-controlled explosion release system or pulsed delivery system according to claim 1, wherein said degradable hydrogel is based on a synthetic polymer backbone from a substantially non-immunogenic polymer.

9. A time-controlled explosion release system or pulsed delivery system according to claim 1, wherein said degradable oligomer or polymer is selected from the group consisting of poly(alkylene glycol), copolymers of ethylene glycol and propylene glycol, poly(oxyethylated polyol), poly(olefinic alcohol), poly(vinylpyrrolidone), poly (hydroxypropyl-methacrylamide), poly(α-hydroxy acid), poly(vinyl alcohol), polyphosphazenes, polyoxazolines; polymers and copolymers of lactones; hydroxy-terminated polyorthoesters; polyacetals; and macromers based on said polymers and further including one or more polymerizable region(s) containing polymerizable end groups.

10. A time-controlled explosion release system or pulsed delivery system according to claim 1, wherein the oligomer or polymer concentration in the aqueous solution is from 15 to 30% by weight.

11. A time-controlled explosion release system or pulsed delivery system according to claim 1, wherein said bio-agent is selected from the group consisting of therapeutic drugs and synthetic molecules, proteins, nucleic acids, vitamins, hormones, nutrients, aromas, fertilisers, anti-microbial agents, insecticides, fungicides, herbicides and pesticides.

12. A time-controlled explosion release system or pulsed delivery system according to claim 1, wherein said bio-agent is present in the core in the form of micro- or nanoparticles.

13. A time-controlled explosion release system or pulsed delivery system according to claim 1, wherein said bio-agent is intimately admixed with or entrapped in said swelling agent.

14. A time-controlled explosion release system or pulsed delivery system according to claim 1, wherein multiple pulsed delivery or multiple explosion release of a bio-agent is effected by providing a mixture of at least two swelling agents having different degradation rates to result in two different lag times for said bio-agent.

15. A time-controlled explosion release system or pulsed delivery system according to claim 1, wherein said lag time ranges from 1 hour to 2 weeks.

16. A time-controlled explosion release system or pulsed delivery system according to claim 1, wherein said semi-permeable membrane is based on cellulose.

17. A time-controlled explosion release system or pulsed delivery system according to claim 1, wherein said semi-permeable membrane comprises two or more polyelectrolyte layers.

18. A time-controlled explosion release system or pulsed delivery system according to claim 1, wherein said semi-permeable membrane comprises a lipid coating surrounding the swelling agent of said core.

19. A time-controlled explosion release system or pulsed delivery system according to claim 1, wherein said degradable polymer hydrogel is positively or negatively charged.

20. A degradable polymer hydrogel being positively or negatively charged and being further coated by means of two or more layers of one or more polyelectrolytes.

21. A degradable oligomer or polymer hydrogel according to claim 20, wherein said degradable hydrogel is a degradable modified dextran.

22. A degradable oligomer or polymer hydrogel according to claim 20, wherein polyelectrolyte is selected from the group consisting of pH dependent cationic polyelectrolytes, pH independent polyelectrolytes and anionic polyelectrolytes.

23. A method of protecting plants or crops by releasing a bio-agent selected from the group consisting of fertilisers, anti-microbial agents, insecticides, fungicides, herbicides and pesticides onto said plants or crops, wherein said bio-agent is included in a time-controlled explosion bio-agent release system or pulsed bio-agent delivery system comprising at least (i) an outer semi-permeable membrane wherein bio-agent release or delivery is caused by explosion of said semi-permeable membrane and begins after a lag time and at least (ii) a core comprising a bio-agent and a swelling agent, wherein said swelling agent is a degradable oligomer or polymer aqueous solution or hydrogel wherein degradation occurs by cleavage of the polymer backbone and/or, in the case of a hydrogel, by cleavage of cross-linking bonds within said hydrogel.
